(19) United States
(12) Patent Application Publication
(10) Pub. No.: US 2006/0281193 A1
(43) Pub. Date: Dec. 14, 2006

(54) Non-optical reading of test zones

(76) Inventors: John F. Petrilla, Palo Alto, CA (US); Carol T. Schembri, San Mateo, CA (US); Peter R. Robrish, San Francisco, CA (US)

Correspondence Address: AVAGO TECHNOLOGIES, LTD., P.O. BOX 1920, DENVER, CO, 80201-1920, US

(21) Appl. No.: 11/148,623

(22) Filed: Jun. 9, 2005

(51) Int. Cl. C12M 1/34; G01N 33/558

(52) U.S. Cl. 436/514; 435/287.2

(57) ABSTRACT

A test zone is read. Conjugate material is exposed to a sample. The conjugate material, when conjugated with at least one analyte in the sample, forms either electrically detectable conjugated material or magnetically detectable conjugated material. Conjugated material in the sample is captured in a test zone. A test is performed on the conjugated material captured in the test zone in order to detect analyte in the sample. The test is an electrical measurement, or a magnetic measurement.

### BACKGROUND

Lateral flow assay test strips are useful to identify the presence of a specific analyte in a sample. Typically during a test, test zones, for example, assay stripes on the test strip, change appearance based on the presence or absence of the specific analyte in the sample. The test zones are then read by a human eye or an imaging system to determine whether the analyte was present in the sample. For more information on the performance of lateral flow assays, see, for example U.S. Pat. No. 6,136,610.

While effective, use of optical reading of the test zone requires the presence of a human tester or sophisticated imaging system. It is desirable to provide alternative systems to read assay stripes in other ways to increase flexibility in designing test systems and to reduce costs.

### SUMMARY OF THE INVENTION

In accordance with an embodiment of the present invention, a test zone is read. A sample is exposed to conjugate material. The conjugate material, when conjugated with at least one analyte in the sample, forms either electrically detectable conjugated material or magnetically detectable conjugated material. Conjugated material in the sample is captured in a test zone. A measurement is performed on the conjugated material captured in the test zone in order to detect analyte in the sample. The measurement is an electrical measurement or a magnetic measurement.

### BRIEF DESCRIPTION OF THE DRAWINGS

FIG. 1 shows a simplified side view of a lateral flow assay test strip used when performing non-optical reading of test zones in accordance with an embodiment of the present invention.

FIG. 2 shows a simplified top view of a lateral flow assay test strip used when performing non-optical reading of test zones in accordance with an embodiment of the present invention.

FIG. 3 and FIG. 4 illustrate conjugation and capturing of an analyte during a test using a lateral flow assay test strip in preparation for performing non-optical reading of test zones in accordance with an embodiment of the present invention.

FIG. 5 is a simplified block diagram showing circuitry used to perform non-optical reading of test zones in accordance with an embodiment of the present invention.

FIG. 6, FIG. 7 and FIG. 8 show bridge circuits useful for reading test zones in accordance with an embodiment of the present invention.

FIG. 9 shows a capacitance comparison circuit configured to read test zones in accordance with an embodiment of the present invention.

FIG. 10 shows a frequency comparison circuit configured to read test zones in accordance with an embodiment of the present invention.

FIG. 11 shows a fringe field capacitor configured to read test zones in accordance with an embodiment of the present invention.

FIG. 12 shows a magnetic reader configured to read test zones in accordance with an embodiment of the present invention.

FIG. 13 shows a test strip with test zones connected in a daisy chain to facilitate non-optical reading of test zones in accordance with an embodiment of the present invention.

FIG. 14 shows a test strip with test zones arranged for reading of test zones using permittivity attributes or permeability attributes in accordance with an embodiment of the present invention.

### DESCRIPTION OF THE EMBODIMENT

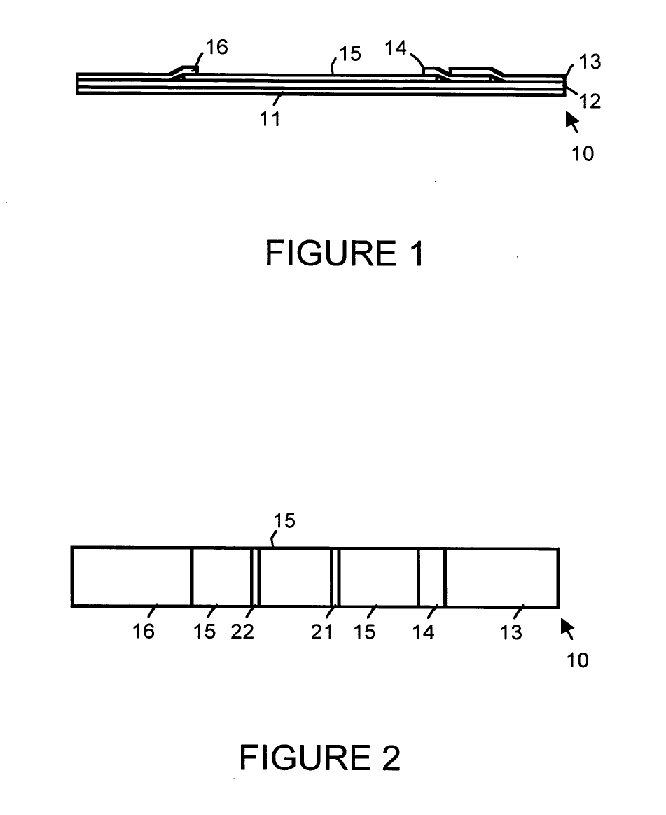

FIG. 1 shows a simplified side view of a lateral flow assay test strip 10. Lateral flow assay test strip 10 includes a backing 11, a pressure sensitive adhesive 12, a sample pad 13, a conjugate pad 14, a membrane 15 and an absorbent pad 16. For example, membrane 15 is composed of nitrocellulose.

FIG. 2 shows a simplified top view of lateral flow assay test strip 10. The top view of lateral flow assay test strip 10 shows the existence of a capture test zone 21 and a control zone 22.

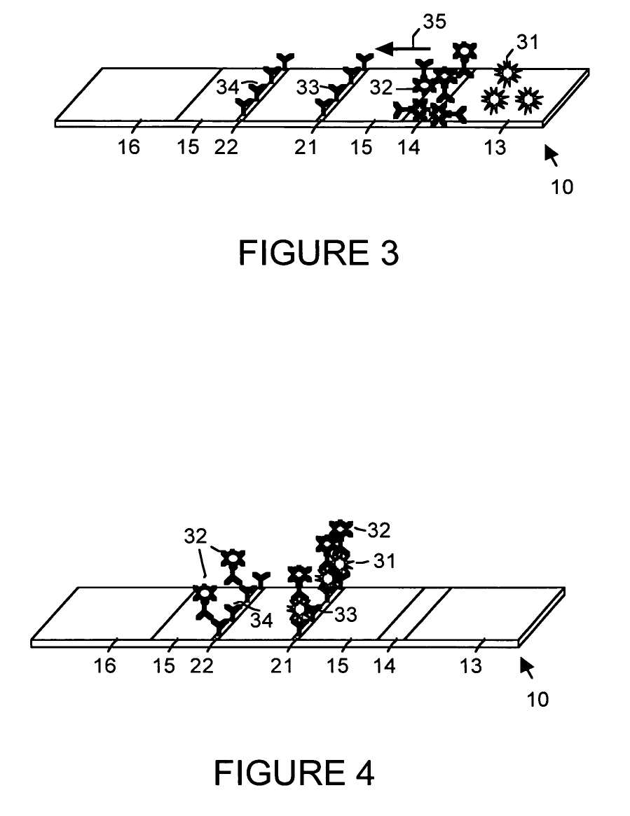

FIG. 3 and FIG. 4 illustrate conjugation and capturing of an analyte during a test using lateral flow assay test strip 10. As shown in FIG. 3, before testing, analyte 31 is present at sample pad 13. Tags 32 are present on conjugation pad 14. Tags 32 include, for example, a first type of antibody and a tag particle. The first type of antibody attaches to analyte 31. For example, the tag particle is a gold particle or some other particle with desired electrical/magnetic properties. Antibodies 33 attached to test zone 21 also are the first type of antibody and also attach to analyte 31. Control structures 34 are a second type of material and are attached to control zone 22. The second type of material selected as control structures 33 attaches to the first type of antibody. For example, the second type of material is composed of antigens, or another type of material that attaches to the first type of antibody. Arrow 35 shows a direction of capillary flow for analyte 31.

FIG. 4 shows molecules of analyte 31 becoming attached to some of the first type of antibodies within tags 32 to form conjugated material. The conjugated material is captured by antibodies 33 attached to test zone 21. The unused tags 32 flow to control structures 34 and are captured by the second type of material that form control structures 34.

For example, FIG. 3 and FIG. 4 are representative of immunoassays. With immunoassays, a higher concentration of analyte normally leads to a stronger signal being detected from capture line 21. On the other hand, with competitive immunoassays, a higher concentration of analyte normally leads to a weaker signal being detected from capture line 21.

Many known methods are available for measurements of resistance, capacitance, complex impedance as well as dielectric constant, permittivity attributes and permeability attributes including measurements of absolute and relative or differential values. For example, measurements can be made using instruments such as Agilent LCR Meter 4294A in combination with Dielectric Test Fixture 16451B, both available from Agilent Technologies, Inc. Measurements can also be incorporated into integrated circuits, examples being the ADXL203 accelerometer, available from Analog Devices, Inc., and the AD7745 Capacitance-to-Digital Converter, also available from Analog Devices, Inc.

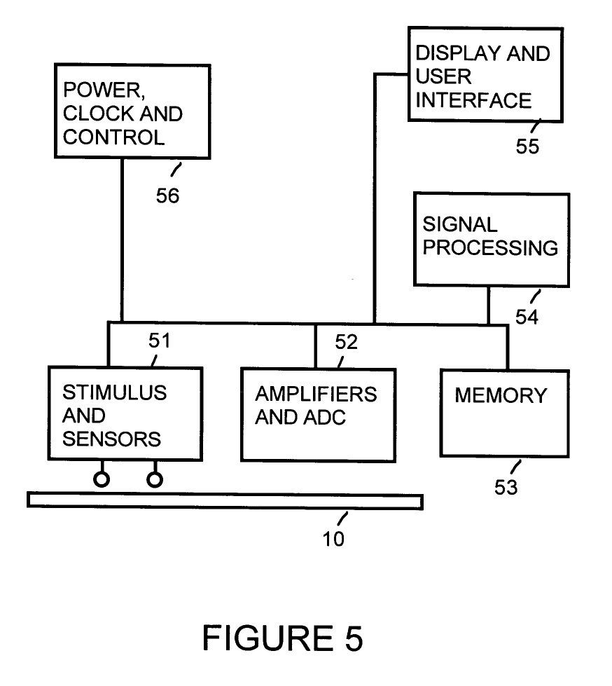

FIG. 5 is a simplified block diagram showing circuitry used to perform non-optical reading of test zones, such as lateral flow assay test strip 10. The circuitry includes stimulus and sensors 51, amplifiers and analog-to-digital conversion (ADC), memory 53, signal processing 54, display and user interface 55 and power, clock and control 56.

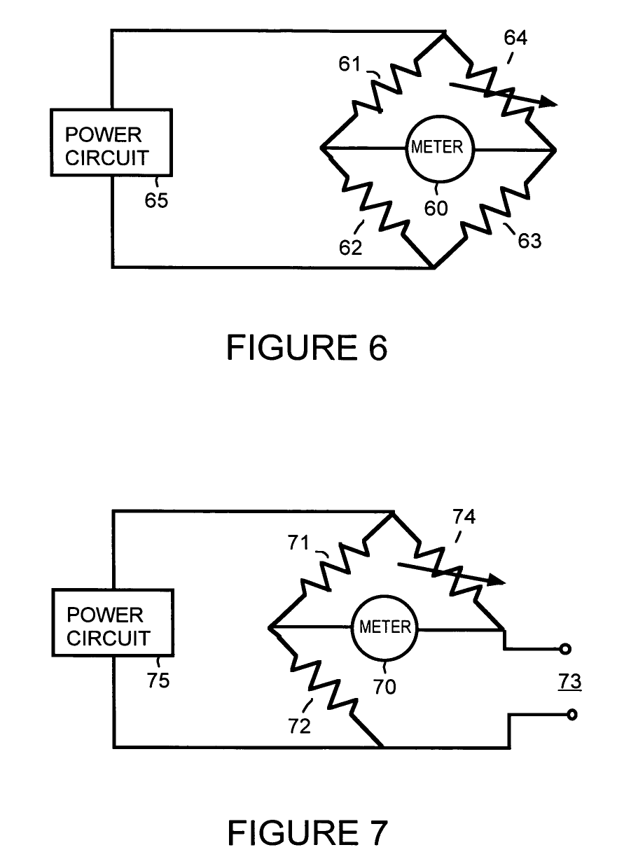

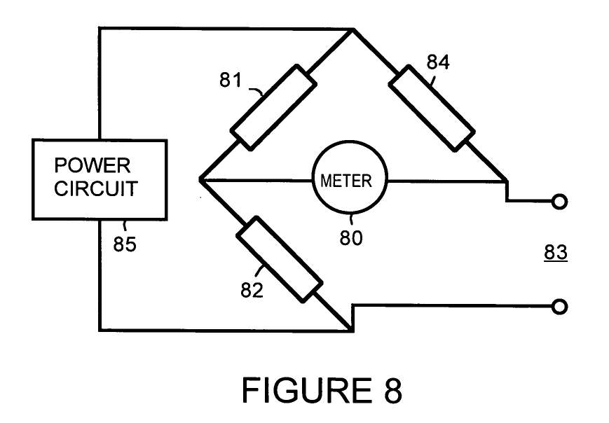

Within stimulus and sensors 51, circuitry is used that is able to make very sensitive measurements. For example, when impedances are measured, various types of bridge circuits can be used for measurement. FIG. 6, FIG. 7 and FIG. 8 give examples of bridge circuits that can be implemented within stimulus and sensors 51.

FIG. 6 shows a power circuit 65, a meter 60, a resistor 61, a resistor 62, a resistor 63 and a variable resistor 64. Variable resistor 64 is varied until meter 60 detects a null value. The unknown value of resistor 63 can be determined from the fixed values of resistor 61 and resistor 62 and from the variable value of variable resistor 64.

Similarly, FIG. 7 shows a power circuit 75, a meter 70, a resistor 71, a resistor 72, and a variable resistor 74. Gap 73 is formed by a gap between two contacts, and represents an unknown value to be detected. Variable resistor 74 is varied until meter 70 detects a null value. The unknown value can be determined from the fixed values of resistor 71 and resistor 72 and from the variable value of variable resistor 74.

FIG. 8 shows a power circuit 85, a meter 80, a complex impedance 81, a complex impedance 82, and a variable complex impedance 84. Gap 83 is formed by a gap between two contacts, and represents an unknown value to be detected. Variable complex impedance 84 is varied until meter 80 detects a null value. The unknown value can be determined from the fixed values of complex impedance 81 and complex impedance 82 and from the variable value of variable complex impedance 84.

Variations or derivatives of the circuitry shown in FIG. 5, FIG. 6, FIG. 7 and FIG. 8 can be configured to provide differential measurements.

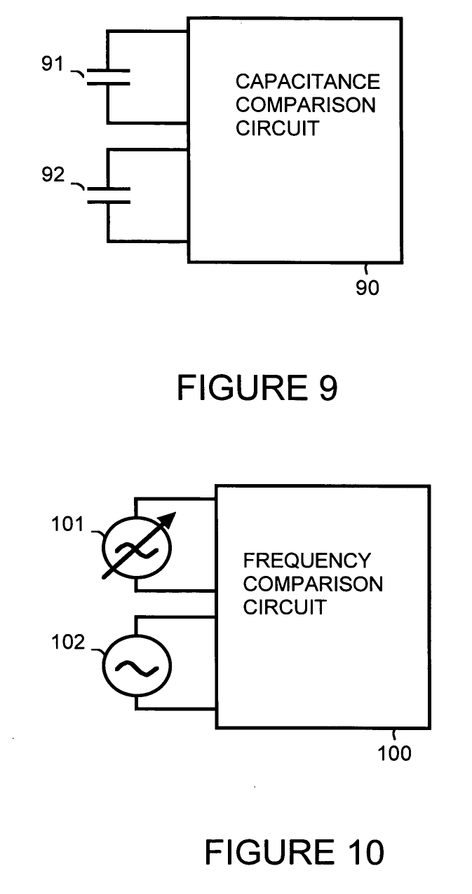

Stimulus and sensors 51, shown in FIG. 5, can also be configured to sense capacitance, rather than resistance. For example, FIG. 9 shows a capacitance comparison circuit 90. A capacitance 91 represents, for example, capacitance measured that includes a capture test zone. A capacitance 92 represents, for example, capacitance measured that includes a control zone.

Resistance, capacitance or complex impedance can be used to control an oscillating signal where one or more oscillation signal characteristics, such as amplitude frequency, phase and/or loss characteristics, can be measured or compared by stimulus and sensors 51, shown in FIG. 5. FIG. 10 shows a frequency comparison circuit 90 that utilizes a variable oscillator 101 and a reference oscillator 102. Measurement of frequency often offers the highest degree of resolution or sensitivity. When utilizing frequency measurement, a capture test zone is used as part of a capacitor to control variable oscillator 101. Measurement of the frequency generated by oscillator 101 can then be used to detect analyte, for example, detecting analyte presence, analyte absence and/or analyte concentration in a sample. Optionally, the signal from oscillator 101 can be combined with the frequency of reference oscillator 102 and the difference can be observed as a third frequency which is often referred to as a beat frequency. Observation of the beat frequency is particularly useful when the change in frequency of oscillator 101 relative to reference oscillator 102 is small.

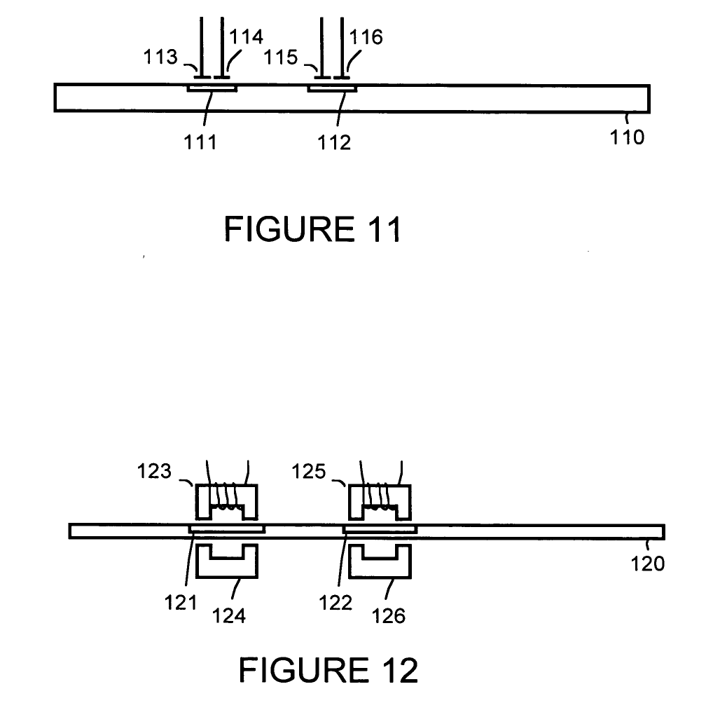

FIG. 11 shows fringe field capacitors configured as sensor elements to read test zones. A first fringe field capacitor consists of an electrode 113, an electrode 114 and dielectric material in an assay stripe 111 on a test strip 110. A second fringe field capacitor consists of an electrode 115, an electrode 116 and dielectric material in an assay stripe 112. Parallel plate capacitors where the test zones are sandwiched between electrodes can also be used as sensor elements.

Fringe capacitors are useful, for example, when the detector or indicator tag is colloidal metal, e.g. gold, or other materials with dielectric characteristics significantly different than the test strip. In this case, accumulation or depletion of the colloidal metal in a test zone (e.g., an assay stripe) can be detected as a change in the characteristics of the dielectric element of a capacitor formed between electrodes placed in proximity with the zone. This may be seen as a change in the effective dielectric constant or as a change in the loss characteristic. Normally a capacitor is viewed as a parallel plate device with the dielectric sandwiched between the plates. However, electric fields fringing around the ends of the plates will form a fringe capacitor involving nearby dielectric material. The expression (Capacitance=Dielectric Constant×Area/Spacing) can be adapted for both using an Effective Area to account for the fringing effect.

One advantage with capacitance or complex impedance measurements is that direct contact with the test strip can be avoided. Since in the assay the dielectric characteristics of the strip is expected to change due to wetting by the test solution, a reference is established by the control zone and the difference between the control zone and test zone becomes the measurement of interest. The control zone can be made to be a fixed concentration of the indicator tags that are immobilized. Here the concentration level can be used to set a threshold. The control zone can also support adjustment for non-specific binding. The change in the control zone from wetting can be used to indicate progress and/or completion of the assay.

In an alternative embodiment, the control zone can be used to collect the tags that have not combined with analyte. Here the concentration of the tags in the test zone (Ctz) and the tags in the control zone (Ccz) can be expected to sum approximately to the initial concentration of tags in the conjugate pad (Ccp,) and the relative concentration ratio (Ctz/Ccz) can be a sensitive indicator of the presence of the analyte.

FIG. 12 shows another embodiment where, instead of dielectric constant (or permittivity attribute) based measurements, permeability (or magnetic properties) based measurements are utilized. In FIG. 12, a conductor is wrapped around a top core 123 and excited with current to produce a magnetic flux. A gap that includes air and a test strip 120 sits between top core 123 and a bottom core 124. Bottom core 124 is optional. The accumulation or depletion of metal particles in test zone 121 will change the reluctance and flux density in the magnetic circuit that includes top core 123, bottom core 124 and the gap between them.

As seen in FIG. 12, permeability attribute measurements also can be made utilizing a control zone 122. A gap that includes air and a test strip 120 sits between top core 125 and a bottom core 126. Bottom core 126 is optional. The accumulation or depletion of metal particles in control zone 122 will change the reluctance and flux density in the magnetic circuit that includes top core 125, bottom core 126 and the gap between them. Magnetic elements can also be used to control the frequency of oscillators

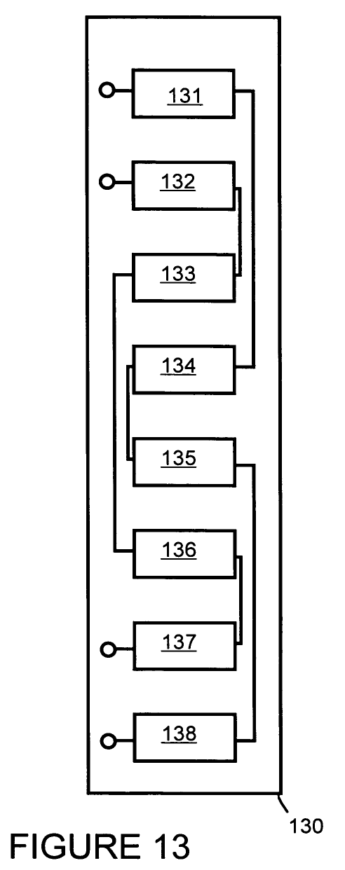

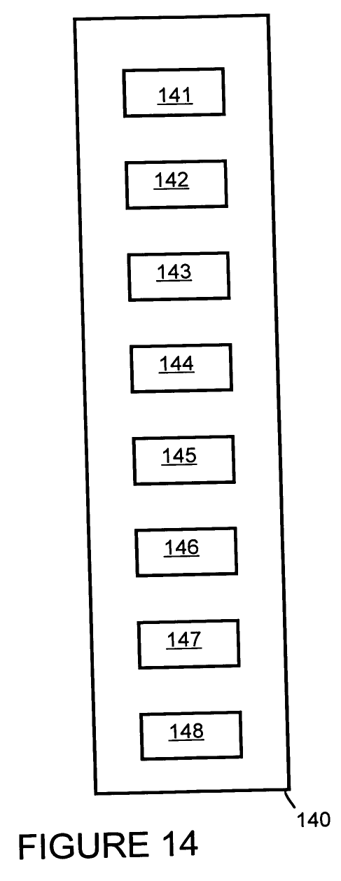

FIG. 13 shows a test strip with test zones connected in a daisy chain to facilitate reading of test zones. A first daisy chain includes a test zone 131, a test zone 134, a test zone 135 and a test zone 138, all placed on a test strip 130. A second daisy chain includes a control zone 132, a control zone 133, a control zone 136 and a control zone 137, all placed on test strip 130. Daisy chains are useful when determination of the presence or absence of one or more of several analytes is desired. This is indicated by breaking the chain by depleting any one of the test zones. Simple resistance measurement requires direct contact to the ends of the daisy chain by the sensor circuit. Alternatively, capacitive coupling to the ends of the daisy requires measurement of complex impedances, but avoids direct contact. The particular arrangement shown in FIG. 13 is suitable for detecting the presence of one or more of several analytes as is useful in drug tests.

Where a change in permittivity attributes or permeability attributes is used no connection is needed between test zones and control zones and no direct contact is required by the sensor circuits. This is illustrated by FIG. 14. FIG. 14 shows a test zone 141, a test zone 143, a test zone 145 and a test zone 147, all placed on a test strip 140. Test strip 140 also includes a control zone 142, a control zone 144, a control zone 146 and a control zone 148.

The foregoing discussion discloses and describes merely exemplary methods and embodiments of the present invention. As will be understood by those familiar with the art, the invention may be embodied in other specific forms without departing from the spirit or essential characteristics thereof. Accordingly, the disclosure of the present invention is intended to be illustrative, but not limiting, of the scope of the invention, which is set forth in the following claims.

We claim:

1. A method for reading test zones comprising:
exposing conjugate material to a sample, the conjugate material when conjugated with at least one analyte within the sample forms one of the following types of conjugated material:
electrically detectable conjugated material,
magnetically detectable conjugated material;
capturing in a test zone, conjugated material that results from the sample being exposed to the conjugate material; and,
performing at least one of the following types of measurements on the conjugated material captured in the test zone in order to detect analyte:
an electrical measurement,
a magnetic measurement.

2. A method as in claim 1 wherein the conjugated material is electrically detectable conjugated material and the type of measurement to detect analyte is an electrical measurement that measures impedances.

3. A method as in claim 1 wherein the conjugated material is electrically detectable conjugated material and the type of measurement to detect analyte is an electrical measurement that measures permittivity attributes.

4. A method as in claim 1 wherein detecting analyte includes determining analyte concentration in the sample.

5. A method as in claim 1 wherein the conjugated material is magnetically detectable conjugated material and the type of measurement to detect analyte is a magnetic measurement that measures permeability attributes.

6. A method as in claim 1 wherein the conjugated material is electrically detectable conjugated material and the type of measurement to detect analyte is an electrical measurement that measures impedances of a daisy chain of test zones.

7. A method as in claim 1 wherein the conjugated material is electrically detectable conjugated material and the type of measurement to detect analyte is a measurement where permittivity or permeability attributes control a measured oscillation signal characteristic.

8. A method as in claim 1 wherein when performing one of the types of measurements to detect analyte, at least one control zone is used for comparative measurements.

9. A testing system comprising:
conjugate material that when conjugated with analyte forms one of the following types of conjugated material:
electrically detectable conjugated material,
magnetically detectable conjugated material;
a test zone in which are captured conjugated material formed when the conjugate material is conjugated with the analyte; and,
measurement circuitry that performs at least one of the following types of measurements on the captured conjugated material in the test zone in order to detect analyte:
an electrical measurement,
a magnetic measurement.

10. A testing system as in claim 9 additionally comprising a control zone used for comparative measurements.

11. A testing system as in claim 9 wherein the conjugated material is electrically detectable conjugated material and the type of measurement to detect analyte is an electrical measurement that measures impedances.

12. A testing system as in claim 9 wherein the conjugated material is electrically detectable conjugated material and the type of measurement to detect analyte is an electrical measurement that measures permittivity.

13. A testing system as in claim 9 wherein the conjugated material is electrically detectable conjugated material and the type of measurement to detect analyte is an electrical measurement that measures capacitance using a fringe capacitor.

14. A testing system as in claim 9 wherein the conjugated material is magnetically detectable conjugated material and the type of measurement to detect analyte is a magnetic measurement that measures permeability attributes.

15. A testing system as in claim 9 wherein the conjugated material is electrically detectable conjugated material and the type of measurement to detect analyte is an electrical measurement that measures impedances through a daisy chain of test zones.

16. A testing system as in claim 9 wherein the conjugated material is electrically detectable conjugated material and the type of measurement to detect analyte is a measurement where permittivity or permeability attributes control a measured oscillation signal characteristic.

17. A testing system as in claim 9 wherein the conjugate material conjugates with additional analytes allowing testing for multiple analytes.

18. A testing system as in claim 9 wherein the conjugate material is stored in a conjugate pad of a test strip.

19. A testing system as in claim 9 wherein the test zone is a capture line on a test strip.

20. A testing system as in claim 9 additionally comprising a control zone used for comparative measurements;
wherein the test zone is a capture line on a test strip; and,
wherein the control zone is a control line on the test strip.
